(19) United States
(12) Patent Application Publication
(10) Pub. No.: US 2019/0314408 A1
(43) Pub. Date: Oct. 17, 2019

(54) USE OF SOMATIC STEM CELLS FOR REDUCING IL-6 LEVEL

(71) Applicant: StemBios Technologies, Inc., Covina, CA (US)

(72) Inventor: James Wang, Monterey Park, CA (US)

(21) Appl. No.: 16/473,217

(22) PCT Filed: Dec. 21, 2017
(86) PCT No.: PCT/US17/67867

§ 371 Date: Jun. 24, 2019

Related U.S. Application Data
(60) Provisional application No. 62/438,699, filed on Dec. 23, 2016.

(51) Int. Cl. A61K 35/12; A61K 35/19; A61K 9/00; A61P 3/10

(52) U.S. Cl. CPC A61K 35/12; A61K 35/19; A61K 2035/124; A61P 3/10; A61K 9/0019

(57) ABSTRACT

A method of decreasing IL-6 level in a subject, comprising: identifying a subject in need thereof; and administering to the subject a composition that contains small cells that are greater than 2 micrometer s and less than 6 micrometer s in size; wherein the small cells include somatic stem cells that are (i) pluripotent; and (i) CD349(+), CD9(−), Oct4(+), Nanog(−), Lgr5(+), CD66e(+), CD133(+), or CD34(+).

### CROSS REFERENCE TO RELATED APPLICATION

This application claims priority to U.S. Provisional Application No. 62/438,699, filed on Dec. 23, 2016, the content of which is hereby incorporated by reference herein in its entirety.

### BACKGROUND

Interleukin 6 )IL-6)is an interleukin that functions in inflammation and maturation of B cells. The protein is mainly produced at sites of acute and chronic inflammation, where it is secreted to induce transcriptional inflammatory responses. IL-6 is implicated in a wide variety of inflammatory disorders such as diabetes and rheumatoid arthritis.

Type 1 diabetes results from the pancreas's failure to produce enough insulin. The lack of insulin results in high blood sugar levels. The cause of type I diabetes is unknown. It is generally believed to involve genetic and environmental factors that lead to an autoimmune response towards beta cells. Lifelong insulin therapy is required for survival.

### SUMMARY

In one aspect, described herein is a method of decreasing IL-6 level in a subject. The method includes identifying a subject in need thereof; and administering to the subject a composition that contains small cells that are greater than 2 micrometers and less than 6 micrometers in size; wherein the small cells include somatic stem cells that are (i) pluripotent; and (i) CD349(+), CD9(+), Oct4(+), Nanog(+), Lgr5(+), CD66e(+), CD133(+), or CD34(+).

In some embodiments, the identifying step includes detecting an increased IL-6 level, as compared to a control level, in a biological sample obtained from the subject. The identified subject can have a condition associated with elevated IL-6 level or condition that can be treated by decreasing IL-6 level (e.g., using an IL-6 antagonist). For example, the subject can have type I diabetes.

The small cells in the composition administered to the subject can further include platelets. For example, 75% to 85% of the small cells can be platelets and 20% to 25% of the small cells can be the somatic stem cells. In some embodiments, the composition contains 10 million to 500 million of the somatic stem cells.

In some embodiments, the composition is prepared by a process that includes: providing a mixture that contains a blood sample obtained from the subject or a donor subject and a divalent cation chelating agent; storing the mixture at a temperature between 2° C. and 12° C. for 3 to 72 hours, whereby the mixture separates into an upper layer and a lower layer, wherein the upper layer contains the population of small cells; and collecting the upper layer, whereby the composition is prepared. 1.5 to 2.0 mg of the divalent cation chelating agent per millimeter of the blood sample can be mixed with the blood sample to obtain the mixture. Preferably, the divalent cation chelating agent is EDTA.

Optionally, prior to obtaining the blood sample to prepare the composition, an action for increasing stem cell number is performed on the subject or donor subject. The action can be administration of an effective amount of fucoidan or a granulocyte-colony stimulating factor.

The process for preparing the composition can further include, after collecting the upper layer, adding a pharmaceutically acceptable excipient to the collected upper layer. Alternatively, the process can further include, after collecting the upper layer, centrifuging the upper layer to obtain a cell pellet. The pellet can be further washed and suspended in a pharmaceutically acceptable excipient. The pharmaceutically acceptable excipient can be one that is free of divalent ions. In some embodiments, the pharmaceutically acceptable excipient is a saline solution.

In some embodiments, the method further includes, after the administering step, detecting IL-6 level in a biological sample obtained from the subject after the administering step.

In another aspect, described herein is a composition for decreasing the level of IL-6 in a subject. The composition contains small cells that are greater than 2 micrometers and less than 6 micrometers in size, wherein the small cells include somatic stem cells that are (i) pluripotent; and (i) CD349(+), CD9(+), Oct4(+), Nanog(+), Lgr5(+), CD66e(+), CD133(+), or CD34(+).

The details of one or more embodiments are set forth in the accompanying drawing and the description below. Other features, objects, and advantages of the embodiments will be apparent from the description and drawing, and from the claims.

### BRIEF DESCRIPTION OF DRAWINGS

FIG. 1 is a graph showing the levels of IL-6 and HbA1c in a type I diabetic patient treated with a SB cell composition.

### DETAILED DESCRIPTION

It was unexpectedly discovered that a composition containing certain small somatic stem cells (e.g., Lgr5(+) or CD349(+) cells) can decrease IL-6 level in a subject. Accordingly, described herein are methods and compositions for decreasing IL-6 level in a subject, for treating conditions associated with an elevated IL-6 level, or for treating conditions that can be treated by an IL-6 antagonist.

### Somatic Stem Cells

There are various types of somatic stem cells, including totipotent stem cells, pluripotent stem cells, multipotent stem cells, and progenitor stem cells (also called unipotent stem cells). Blastomere-like stem cells (BLSCs) are totipotent or pluripotent somatic stem cells. Very small embryonic-like stem cells (VSELs) are pluripotent somatic stem cells. SB cells are pluripotent or multipotent somatic stem cells. Mesenchymal stem cells (MSCs) and hematopoietic stem cell (HSC) are multipotent somatic stem cells.

The size (Z) of a cell, such as a stem cell, as used herein may refer to (1) the conventional definition of the size or representative length of a cell in the field of cell biology or the field of stem cells, (2) the diameter of a cell especially when the cell is substantially spherical, (3) the length of the major axis of a cell especially when the cell is substantially ellipsoidal, (4) the width of a cell when the shape of the cell has an approximate shape of a square, (5) the length of a cell when the shape of the cell has an approximate shape of a rectangle, or (6) the greatest cross-sectional or transverse dimension of a cell. The size (Z), either the diameter, length, width, or greatest cross-sectional or transverse dimension, can be determined or measured, for example, using an image of the cell obtained from an optical microscope or from an electron microscope (e.g., scanning electron microscope (SEM)), or using data (e.g., two-dimensional dot, contour or density plot) of the cell obtained from a flow cytometer. An image of a cell obtained from an optical microscope or electron microscope may be a two-dimensional (2D) cross section or three-dimensional (3D) structure of the cell. As an example, the size (Z) of the cell may be obtained by measuring the greatest cross-sectional or transverse dimension of the cell in a 2D cross-sectional image obtained from an optical microscope or an electron microscope (e.g., SEM).

The term “small cell” (e.g., small somatic stem cell) refers to a cell having a size less than 6 micrometers (e.g., between 2.0 and 6.0 micrometers). The term “large cell” refers to a cell having a size greater than 6 micrometers.

CD349(+) SB cells are pluripotent or multipotent somatic stem cells. CD349(+) SB cells may also be CD9(+), Oct4(+), and Nanog(+), as well as CD133(−), CD90(−), CD34(−), and Sox2(−). CD349(+) SB cells each have a size equal to or less than 4, 5 or 6 micrometers, such as between 0.1 and 6.0 micrometers, between 0.5 and 6.0 micrometers, between 1.0 and 6.0 micrometers, between 2.0 and 6.0 micrometers, between 0.1 and 5.0 micrometers, between 0.5 and 5.0 micrometers, between 1.0 and 5.0 micrometers, between 0.1 and 4.0 micrometers, between 0.5 and 4.0 micrometers, or between 1.0 and 4.0 micrometers. Preferably, the size is greater than 2 micrometers and less than 6 micrometers.

Lgr5(+) SB cells are also pluripotent or multipotent somatic stem cells. They may also be Oct4(+) and Nanog(+), as well as CD133(−), CD66e(−), CD4(−), CD8(−), CD9(−), CD10(−), CD11(−), CD16(−), CD17(−), CD18(−), CD19(−), CD20(−), CD21(−), CD31(−), CD42(−), CD63(−), CD34(−), Lin(−), CD38(−), CD90(−), CD45(−), CD349(−), and Sox2(−). The size of a Lgr5(+) SB cell can be equal to or less than 4, 5 or 6 micrometers, such as between 0.1 and 6.0 micrometers, between 0.5 and 6.0 micrometers, between 1.0 and 6.0 micrometers, between 2.0 and 6.0 micrometers, between 0.1 and 5.0 micrometers, between 0.5 and 5.0 micrometers, between 1.0 and 5.0 micrometers, between 0.1 and 4.0 micrometers, between 0.5 and 4.0 micrometers or between 1.0 and 4.0 micrometers. Preferably, a Lgr5(+) SB cell is greater than 2 micrometer s and less than 6 micrometer s in size.

Blastomere-like stem cells (BLSCs) are CD66e(+) totipotent or pluripotent somatic stem cells. They can each have a size that is equal to or less than 4, 5 or 6 micrometer s, such as between 0.1 and 6.0 micrometers, between 0.5 and 6.0 micrometers, between 1.0 and 6.0 micrometers, between 2.0 and 6.0 micrometers, between 0.1 and 5.0 micrometers, between 0.5 and 5.0 micrometers, between 1.0 and 5.0 micrometers, between 0.1 and 4.0 micrometers, between 0.5 and 4.0 micrometers or between 1.0 and 4.0 micrometers. For example, a BLSC can have a size that is greater than 2 micrometer s and less than 6 micrometer s.

Very small embryonic-like stem cells (VSELs) are pluripotent somatic stem cells, which can be CD133(+) or CD34(+). A VSEL can also be CD45(−) and Lin(−). For example, a VSEL can be CD133(+), CD45(−) and Lin(−), or CD34(+), CD45(−) and Lin(−). The size of a VSEL can be equal to or less than 4, 5 or 6 micrometer s, such as between 0.1 and 6.0 micrometers, between 0.5 and 6.0 micrometers, between 1.0 and 6.0 micrometers, between 2.0 and 6.0 micrometers, between 0.1 and 5.0 micrometers, between 0.5 and 5.0 micrometers, between 1.0 and 5.0 micrometers, between 0.1 and 4.0 micrometers, between 0.5 and 4.0 micrometers or between 1.0 and 4.0 micrometers. A VSEL can be greater than 2 micrometers and less than 6 micrometer s in size.

Mesenchymal stem cells (MSCs) are multipotent somatic stem cells. An MSC may express one or more of the cell surface markers CD13, CD29, CD44, CD73, CD90 and CD105. MSCs constitute a very heterogeneous population. Some types of MSCs may be equal to or less than 4, 5 or 6 micrometers, such as between 0.1 and 6.0 micrometers, between 0.5 and 6.0 micrometers, between 1.0 and 6.0 micrometers, between 0.1 and 5.0 micrometers, between 0.5 and 5.0 micrometers, between 1.0 and 5.0 micrometers, between 0.1 and 4.0 micrometers, between 0.5 and 4.0 micrometers or between 1.0 and 4.0 micrometers, in size. Other types of MSCs may be greater than 6, 7 or 10 micrometers in size.

Hematopoietic stem cells (HSCs) are multipotent somatic stem cells. They can be CD34(+), cKit(−), CD38(−), Lin(−) cells or CD150(+), CD244(−), and CD48(−) cells. HSCs can be equal to or less than 4, 5 or 6 micrometer s, such as between 0.1 and 6.0 micrometers, between 0.5 and 6.0 micrometers, between 1.0 and 6.0 micrometers, between 0.1 and 5.0 micrometers, between 0.5 and 5.0 micrometers, between 1.0 and 5.0 micrometers, between 0.1 and 4.0 micrometers, between 0.5 and 4.0 micrometers or between 1.0 and 4.0 micrometers in size.

### Actions for Increasing Stem Cells

An action (X) as used herein is an action that may be effective for increasing the number of one or more types of stem cells in vivo, e.g., in a human subject or non-human subject. Actions (X) can include:

1. Taking drugs such as synthetic drugs or compounds derived from nature;

2. Taking herbs or Chinese herbal medicines, such as Cordyceps sinensis, ginseng, Lycium Chinense Mill, Ganoderma lucidum (lingzhi), Taiwanofungus camphoratus, and/or Brazil mushroom;

3. Taking nutrients or dietary supplements, such as nutrition pills or powder, including the following materials or elements: vitamins (Vitamin A, B, B complex, B₁₂, D, D₃, E, etc.), macro and/or trace minerals (e.g., calcium, sodium, potassium, fluorine, bromine, chromium, iodine, silicon, selenium, beryllium, lithium, cobalt, vanadium and/or nickel), polysaccharides, high molecular weight fucose-containing glycoproteins, seaweed (including green algae, blue-green algae, brown algae, and etc.), fucose, fucoidan (a major component of brown algae), oligo fucoidan, algae, brown algae containing fucoidan (for example, brown algae grown and produced in Okinawa, Japan), Japanese Mozuku, green algae, blue-green algae (or blue algae), brown algae (including mozuku, kelp, undaria, sargassum fusiforme, pinnatifida, and etc.), phytochemical (e.g., isoflavones or phytoestrogen), lycopene, epigallocatechin gallate (EGCG), green tea essence, gluconutrients (e.g., Xylose, Galactose, Glucose, Mannose N-acetylglucosamine, N-acetylgalaetosanmine, or N-acetylneuraminic acid), fish oil, China toona (toona sinensis), and/or nutrients extracted from plant, leaf, fruit, vegetable, fish, seaweed, or algae;

4. Practicing a vegetarian dietary;

5. Taking or eating healthy food or organic food;

6. Taking an alternative (non-traditional) medicine;

7. Being subjected to an alternative therapy or treatment such as the Gerson therapy or the Breuss cancer cure;

8. Being subjected to acupuncture;

9. Being subjected to massage such as foot massage;

10. Exercising such as walking, jogging, dancing, gymnastics, Yoga, aerobic exercise, and/or Taijiquan (Chinese shadow exercise);

11. Sleeping (for purpose of measuring the quality of sleep);

12. Meditating;

13. Practicing a health improvement program or a disease curing program designed by an individual, a health professional, or a medical doctor;

14. Taking a certain nutrient for improving health of a certain organ in a body, for example, taking lycopene to improve the health of prostate;

15. Taking a rehabilitation program to heal the injury, or to heal the wounds caused by surgery, or to cure a disease;

16. Taking a medicinal liquor (or called medicinal wine, medicated liquor or medicated wine) made from, e.g., immersing one Chinese medicine or multiple Chinese medicines in liquor or wine for a period of time, such as ginseng wine made from immersing ginseng in a high alcohol concentration rice wine for a month;

17. Taking one or more drugs approved by a government department or authority, such as U.S. food and drug administration (U.S. FDA), for curing a specific disease (e.g., a type of cancer, skin disease, kidney disease and/or so on);

18. Taking or being subjected to a treatment or therapy approved by a government department for curing a specific disease (e.g., a type of cancer, skin disease, or kidney disease);

19. Practicing a religious activity, such as praying for peace or worshiping God;

20. Being exposed directly or indirectly to sunshine or sunlight (in the morning between, for example, 10 minutes before sunrise and 50 minutes after sunrise (containing significant amount of infrared (IR) light); or around noon, for example, between 11:30 AM to 12:30 PM (containing significant amount of ultra-violet (UV) light); or in the afternoon, for example, between 50 minutes before sunset and 10 minutes after sunset (containing significant amount of infrared (IR) light));

21. Being exposed to the lamp light or the light emitting diode (LED) light, which may include a whole spectrum of visible lights, IR light, red light, green light, blue light, or UV light, or a combination of more than one of the above lights;

22. Exercising or being subjected to programs, therapies, methods, apparatus and/or systems for improving body's self-healing, for example, a method or therapy (e.g., Hyperbaric oxygen therapy) performed after injury or surgery for improving self-healing;

23. Drinking coffee such as black coffee;

24. Drinking tea such green tea, black tea, or jasmine tea;

25. Drinking red wine;

26. Taking melatonin;

27. Listening to music such as Mozart's or Beethoven's symphony;

28. Injecting a substance (e.g., a nutrient or supplement) containing fucoidan or oligo fucoidan;

29. Taking hormone supplements or being subjected to a hormone injection;

30. Injecting a granulocyte-colony stimulating factor (G-CSF or GCSF), which is a glycoprotein;

31. Being subjected to a course of GCSF injections; and

32. Taking a nutrient, a nutrient product, a nutrient fluid, a nutrient drink, a nutrient liquid, or a nutrient food containing (1) varieties of amino acids (such as Arginine, Histidine, Lysine, Aspartic acid, Glutamic acid, Serine, Threonine, Asparagine, Glutamine, Cysteine, Valine, Proline, Glycine, Selenocysteine, Alanine, Isoleucine, Leucine, Phenylalanine, Methionine, Tyrosine, or Tryptophan), (2) balanced amino acids, or (3) 9 essential amino acids (i.e., Histidine, Isoleucine, Leucine, Lysine, Methionine, Phenylalanine, Threonine, Tryptophan and Valine) for human bodies. For examples: (a) Product produced or extracted from the fermentation of red, green, black beans; (b) Liquid, fluid, or drink produced from fermentation of a fruit or a combination of fruits, such as sugar beet, apple, guava, kiwi, grape, pineapple, red pitaya (dragon fruit), green papaya, tomato, and/or avocado, etc.; (c) A medicinal liquor (or called medicinal wine, medicated liquor, or medicated wine) made from, e.g., immersing one Chinese medicine or multiple Chinese medicines in liquor or wine for a period of time, such as ginseng wine made from immersing ginseng in a high alcohol concentration rice wine for a month.

### Stem Cell-Containing Composition

A stem cell-containing composition (e.g., a stem cell-containing solution) can be prepared using an exemplary method described below.

An action (X), which may be one of the above-mentioned actions (X), is performed on a subject. The subject, for example, is a human (e.g., child, teenager, adult, or elderly) or a non-human animal. Examples of a non-human animal include a primate (e.g., monkey or gorilla), dog, rodent (e.g., mouse or guinea pig), cat, horse, cow, cattle, sheep, pig, chicken, duck, goose, bird, and elephant.

For example, the subject can ingest a stem cell-mobilization agent such as a fucoidan-containing compound. The fucoidan-containing compound can be a brown algae supplement. A pill of the brown algae supplement contains 80% of a mozuku powder, 15% of crystalline cellulose, 3% of sucrose fatty acid esters, and 2% of micro or fine silica (containing silicon dioxide). The mozuku powder may be extracted from mozuku brown algae (one kind of seaweed) grown in the sea around and near Okinawa, Japan. The mozuku powder is then mixed with crystalline cellulose, sucrose fatty acid esters, and micro or fine silica (containing silicon dioxide) to form the pill of the brown algae supplement, which contains 0.1 grams of fucoidan. The subject may ingest 20 or more pills (e.g., at least 30 pills) of the brown algae supplement or 2 grams or more (such as at least 3 grams) of fucoidan. In another example, the subject may be injected with a granulocyte-colony stimulating factor (GCSF), i.e., a mobilization agent, or may be subjected to a course of GCSF injections.

After an action (X) is performed, the subject waits for a period of time (e.g., a predetermined period of time), such as between 15 minutes and 60 minutes, between 20 minutes and 100 minutes, between 30 minutes and 4 hours, between 60 minutes and 90 minutes, between 0.5 hours and 3 hours, between 1 hour and 6 hours, between 1 hour and 12 hours, between 12 hours and 36 hours, or between 36 hours and 50 hours. Performing action (X) and waiting for a period allow one or more specific types of somatic stem cells, such as SB cells (i.e., CD349(+) and Lgr5(+) SB cells), to be mobilized into the subject's peripheral blood from, e.g., the subject's bone marrow. The peripheral blood of the subject thus becomes enriched with the one or more specific types of somatic stem cells. The one or more specific types of somatic stem cells, for example, may be or may include one or more of the somatic stem cells described above. For instance, the one or more specific types of somatic stem cells may be or may include somatic stem cells less than 6 micrometers in size, and more preferably greater than 2 micrometer s in size, such as CD349(+) somatic stem cells and/or Lgr5(+) somatic stem cells.

Performing action (X) and waiting for a period are optional steps. In other words, to make a stem cell-containing composition, a blood sample can be obtained from a subject without first performing any action (X) on the subject.

Immediately after the above waiting step (if action (X) and the waiting step are carried out), a blood sample is obtained from the peripheral blood of the subject and placed into one or more containers (e.g., a bag, one or more syringes, or one or more tubes) containing a divalent cation chelating agent. The blood sample is mixed with the divalent cation chelating agent in the container to form a mixture. The divalent cation chelating agent, e.g., an anticoagulant, may be ethylenediaminetetraacetic acid (EDTA), such as K2 EDTA anticoagulant or K3 EDTA anticoagulant, having a weight, e.g., greater than 70 mg, such as between 90 and 900 mg, between 120 and 450 mg, or between 150 and 400 mg. Alternatively, the divalent cation chelating agent may be citrate having a weight, e.g., greater than 70 mg, such as between 90 and 900 mg, between 120 and 450 mg, or between 150 and 400 mg. The blood sample contains a plurality of cells, including small cells less than 6 micrometers in size and large cells greater than 6 micrometer s in size. The small cells, for example, contain platelets and small somatic stem cells less than 6 micrometer s in size. For instance, the small somatic stem cells contain the one or more specific types of somatic stem cells (i.e., SB cells, for example), BLSCs (i.e., CD66e(+) somatic stem cells), and VSELs (e.g., CD133(+) somatic stem cells and CD34(+) somatic stem cells). The large cells, for example, contain large somatic stem cells greater than 6 micrometer s in size and lineage cells such as red blood cells and white blood cells. The blood sample may have a volume greater than or equal to 45 milliliters, such as between 60 and 500 milliliters, between 80 and 250 milliliters or between 100 and 200 milliliters. In an example, the blood sample may be mixed with 1.5 mg or more, such as between 1.6 and 2.0 mg, of the divalent cation chelating agent (such as K2 EDTA, K3 EDTA, or citrate) per milliliter of the blood sample to form the mixture in the container.

Next, the mixture is processed to form a stem cell-containing solution. The process can include steps for stem cell activation and purification/isolation. The term “purification” or “isolation” as used herein means substantial separation of small cells (e.g., cells greater than 2 micrometer s and less than 6 micrometer s in size) from large cells (e.g., cells greater than 6 micrometer s in size).

The mixture can be stored at a temperature between 2 degrees Celsius (° C.) and 12° C., more preferably between 2° C. and 7° C. or at 4° C., in a suitable facility (e.g., refrigerator or other device used to keep things cold) for a predetermined period of time. The period of time can be between 3 hours and 72 hours, and more preferably between 3 hours and 6 hours, between 6 hours and 72 hours, between 6 hours and 48 hours, between 16 hours and 72 hours, between 16 hours and 48 hours, between 36 hours and 60 hours, between 48 hours and 72 hours, or around 48 hours. After the mixture has been stored for the predetermined period of time, the one or more specific types of somatic stem cells (e.g., SB cells) in the mixture may be activated by the divalent cation chelating agent (such as K2 EDTA, K3 EDTA, or citrate), i.e., the cell cycle of the one or more specific types of somatic stem cells is activated from G0 into G1. The activation may relate to the ability of the divalent cation chelating agent to repress p53's function (presumably by chelating Zn²⁺), thereby allowing the one or more specific types of somatic stem cells (e.g., SB cells) to exist from the G0 quiescence stage into the G1 stage of the cell cycle. As the p53 protein requires Zn²⁺ to fold properly and form a functional protein, chelating Zn²⁺ by the divalent cation chelating agent may be a key step to activate the one or more specific types of somatic stem cells (e.g., SB cells). It is possible that the divalent cation chelating agent can chelate other divalent ions (e.g., Ca²⁺), thereby activates the one or more specific types of somatic stem cells and forces them to proliferate and expand.

As the mixture is stored, it separates into separate layers including an upper layer and a lower layer due to gravity. The upper layer, or the supernatant, may have a volume between 20 and 250 milliliters, between 40 and 125 milliliters, or between 50 and 100 milliliters. The upper layer contains platelets, serum, and one or more specific types of small somatic stem cells (i.e., SB cells, for example), BLSCs (i.e., CD66e(+) somatic stem cells), and VSELs (e.g., CD133(+) somatic stem cells and CD34(+) somatic stem cells). Most of the large cells containing lineage cells and the large somatic stem cells of the blood sample, such as greater than 95%, 98% or 99% of the large cells of the blood sample, are in the lower layer. The ratio of the volume of the supernatant to the volume of the blood sample, for example, may range from one third to one half.

Next, substantially all of the upper layer may be collected or transferred into a liquid container, such as a bag, a syringe, or a glass bottle, to produce a stem cell-containing solution or stem cell mixture. The upper layer, e.g., a stem cell-containing solution, contains small cells, which include platelets and small somatic stem cells. The number of small somatic stem cells in the stem cell-containing solution can be greater than or equal to 10 million (e.g., greater than or equal to 30 million, greater than or equal to 50 million, between 10 million and 500 million, between 25 million and 300 million, or between 30 million and 500 million). The stem cell-containing solution may also contain the divalent cation chelating agent (e.g., EDTA) and/or growth factors.

In addition, the stem cell-containing solution barely includes or substantially excludes large cells (e.g., large somatic stem cells and lineage cells). For example, large cells can constitute less than 5% (e.g., less than 1%, 0.5%, or 0.01%) of the total number of cells in the stem cell-containing solution. For example, the number of red blood cells in the stem-cell containing solution (e.g., the collected upper layer) can be less than 10⁵ or 10⁴ per milliliter. Preferably, the number of red blood cells per milliliter of the stem cell-containing solution is less than 10³. The number of white blood cells per milliliter of the stem cell-containing solution can be less than 10⁴ (e.g., less than 10³). Preferably, the number of white blood cells per milliliter of the stem-cell containing solution is less than 10².

Greater than 95% (e.g., 99% or 99.99%) of all of the cells in the stem cell-containing solution can be small cells. The small cells can include platelets, Lgr5(+)cells, CD349(+) cells, CD133(+) cells, CD34(+), and CD66e(+) cells. Platelets can constitute 75% to 85% of the small cells in the stem cell-containing solution. Greater than 4% (e.g., greater than 5% or between 4.5% and 10%) of all of the small cells can be Lgr5+ somatic stem cells. CD349(+) somatic stem cells can constitute greater than 4% (e.g., greater than 5% or between 4.5% and 10%) of all of the small cells the stem cell-containing solution. Less than 2% (e.g., less than 1% or 0.5%) of the small cells can be CD133(+) cells and CD34(+) cells combined. Less than 6% (e.g., less than 5% or 4.5%) of the small cells can be CD66e(+) cells.

Any specific small cells can also be further isolated or depleted from the collected upper layer using flow cytometry or other conventional techniques (e.g. antibody-based techniques such as antibody-conjugated beads).

The collected upper layer can be used as is as a stem cell-containing solution (e.g., administered to a subject or stored) or further processed. For example, it can be further purified (e.g., filtered) or mixed with one or more additional components. A suitable cell medium or solution free from Ca²⁺ having a volume, e.g., greater than 400 milliliters, such as between 500 and 900 milliliters, can be added to the collected upper layer to make a stem cell-containing solution. The suitable medium or solution free from Ca²⁺, such as a NaCl-containing solution, may be further free from any divalent ions, including Mg²⁺. The NaCl-containing solution, for example, can be normal saline (e.g., a solution of 0.90% w/v of NaCl, about 300 mOsm/L or 9.0 gram per liter).

The stem cell-containing solution may be stored in a frozen storage temperature, e.g., equal to or less than −70° C. or −80° C. (e.g., between −75° C. and −85° C.) for an extended period of time (e.g., more than one week, one month, or one year). When ready for use, the frozen stem cell-containing solution can be quickly thawed and, optionally, mixed with the aforementioned suitable medium or solution free from Ca²⁺ (e.g., 0.9% NaCl).

### Treatment Method

The stem cell-containing composition produced by the procedure described above can be used to decrease IL-6 level in a subject. For example, it can be used to treat a condition associated with elevated level of IL-6 or a condition that can be treated by an IL-6 antagonist or by decreasing IL-6 level.

IL-6 level can refer to an IL-6 protein level, mRNA level, cDNA level, or functional level in any biological sample, e.g., blood sample, bone marrow sample, urine sample, or solid tissue sample, obtained from a subject. An elevated IL-6 level is a level that is higher than the level or range of levels found in healthy individuals or individuals without a condition associated with elevated IL-6 level. For example, a normal IL-6 level in blood can be about 4.7 pg/ml or lower. Methods of measuring protein, mRNA, cDNA, and functional levels are well known in the art, e.g., ELISA, Western blotting, and real time PCR.

Before the stem cell-containing composition is administered to a subject, whether the subject has an elevated level of IL-6 can be determined. Alternatively or in addition, after the composition is administered, the IL-6 level in the subject can be determined to monitor treatment efficacy and to make treatment decisions. Optionally, a disease parameter or symptom (e.g., HbA1c or glucose level) in the subject can be evaluated before and/or after the administration.

Human and other mammalian IL-6 sequences are known in the art, e.g., NCBI accession nos. NP_000591 (human), NP_001305024 (human), NP_036721 (rat), and NP_112445 (mouse).

The stem cell-containing composition described herein can be administered to a subject in need thereof via any route of administration, e.g., intravenous, intraarticular, conjunctival, intracranial, intraperitoneal, intrapleural, intramuscular, intrathecal, or subcutaneous route of administration. The composition can contain between 10 million and 500 million small somatic stem cells. Autologous or allogeneic somatic stem cells can be used. The composition can be administered to a subject, for example, every 1-14 days, every 2-4 weeks, every 1-6 months, or every 2-12 months, for a treatment period (e.g., 1-36 months or 2-10 years), or whenever needed.

Conditions that can be treated with the stem-cell containing composition include type I diabetes, rheumatoid arthritis, atherosclerosis, and depression.

A “subject” refers to a human or a non-human animal. “Treating” or “treatment” refers to administration of a compound or composition to a subject, who has a disorder, with the purpose to cure, alleviate, relieve, remedy, delay the onset of, or ameliorate the disorder, the symptom of the disorder, the disease state secondary to the disorder, or the predisposition toward the disorder. An “effective amount” refers to an amount of the compound or composition that is capable of producing a medically desirable result in a treated subject. The treatment method can be performed alone or in conjunction with other drugs or therapy.

The specific example below is to be construed as merely illustrative, and not limitative of the remainder of the disclosure in any way whatsoever. Without further elaboration, it is believed that one skilled in the art can, based on the description herein, utilize the present disclosure to its fullest extent.

### EXAMPLE

100 to 150 ml of peripheral blood samples were obtained from one patient who suffered from type I diabetes. EDTA-coated tubes containing the blood samples were stored for 6 to 48 hours at 4° C. until the blood separated into two distinct layers. The top layer, which contained SB cells, was collected and delivered autologously back into the patient through intravenous injection. The patient was administered two treatments.

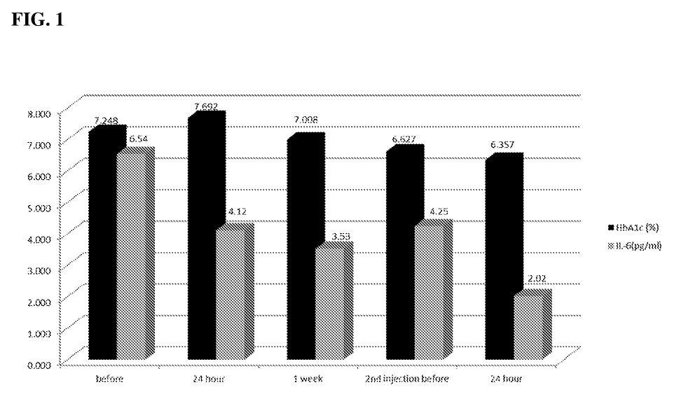

After each treatment, the IL-6 level and hemoglobin A1c (HbA1c) level in the patient were determined. As shown in FIG. 1, both levels decreased after each treatment. Significantly, after the second treatment, the HbA1c level was below 6.5%. See FIG. 1. A person with an HbA1c level higher than 6.5% is considered to have diabetes.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described embodiments, and without departing from the spirit and scope thereof, can make various changes and modifications of the embodiments to adapt it to various usages and conditions. Thus, other embodiments are also within the claims.

What is claimed is:

1. A method of decreasing IL-6 level in a subject, comprising:
identifying a subject in need thereof; and
administering to the subject a composition that contains small cells that are greater than 2 micrometer s and less than 6 micrometer s in size;
wherein the small cells include somatic stem cells that are (i) pluripotent; and (i) CD349(+), CD9(+), Oct4(+), Nanog(+), Lgr5(+), CD66e(+), CD133(+), or CD34(+).

2. The method of claim 1, wherein the identifying step includes detecting an increased Il-6 level, as compared to a control level, in a biological sample obtained from the subject.

3. The method of claim 2, wherein the biological sample is a blood sample.

4. The method of claim 1, wherein the subject has a condition associated with an increased IL-6 level or condition that can be treated by an IL-6 antagonist.

5. The method of claim 4, wherein the subject has type I diabetes.

6. The method of any of claims 1-5, wherein the small cells further include platelets.

7. The method of claim 6, wherein, 75% to 85% of the small cells are the platelets and 20% to 25% of the small cells are the somatic stem cells.

8. The method of claim 7, wherein the composition contains 10 million to 500 million of the somatic stem cells.

9. The method of claim 8, wherein the composition is prepared by a process that includes:
providing a mixture that contains a blood sample obtained from the subject or a donor subject and a divalent cation chelating agent;
storing the mixture at a temperature between 2° C. and 12° C. for 3 to 72 hours, whereby the mixture separates into an upper layer and a lower layer, wherein the upper layer contains the population of small cells; and
collecting the upper layer, whereby the composition is prepared.

10. The method of claim 9, wherein the divalent cation chelating agent is EDTA.

11. The method of claim 10, wherein 1.5 to 2.0 mg of divalent cation chelating agent per millimeter of the blood sample is mixed with the blood sample to obtain the mixture.

12. The method of claim 10, wherein, prior to obtaining the blood sample, an action for increasing stem cell number is performed on the subject or donor subject.

13. The method of claim 12, wherein the action is administration of an effective amount of fucoidan or a granulocyte-colony stimulating factor.

14. The method of claim 10, wherein the process for preparing the composition further includes, after collecting the upper layer, adding a pharmaceutically acceptable excipient to the collected upper layer.

15. The method of claim 10, wherein the process for preparing the composition further includes, after collecting the upper layer, centrifuging the upper layer to obtain a cell pellet.

16. The method of claim 15, wherein the process for preparing the composition further includes washing the pellet and suspending the pellet in a pharmaceutically acceptable excipient.

17. The method of claim 15 or 16, wherein the pharmaceutically acceptable excipient is free of divalent ions.

18. The method of claim 17, wherein the pharmaceutically acceptable excipient is a saline solution.

19. The method of any of claims 1-18, wherein the composition is administered intravenously.

20. The method of any of claims 1-19, further comprising, after the administering step, detecting an IL-6 level in a biological sample obtained from the subject after the administering step.
